(19) United States
(12) Patent Application Publication
(10) Pub. No.: US 2023/0022378 A1
(43) Pub. Date: Jan. 26, 2023

(54) METHODS FOR THE PREPARATION OF ETHYL 3-BROMO-1-(3-CHLOROPYRIDIN-2-YL)-1H-PYRAZOLE-5-CARBOXYLATE

(71) Applicants: FMC Corporation, Philadelphia, PA (US); FMC Agro Singapore Pte. Ltd, Singapore (SG)

(72) Inventors: Shuren Ji, Shanghai (CN); Jie Luan, Shanghai (CN); Jianhua Mao, Shanghai (CN); Hao Wang, Shanghai (CN); YiHui Xu, Shanghai (CN)

(21) Appl. No.: 17/775,434

(22) PCT Filed: Nov. 11, 2020
(86) PCT No.: PCT/US2020/059923

§ 371 Date: May 9, 2022

Related U.S. Application Data
(60) Provisional application No. 62/933,553, filed on Nov. 11, 2019.

(51) Int. Cl. C07D 401/04

(52) U.S. Cl. CPC C07D 401/04

(57) ABSTRACT

Described herein are novel methods of synthesizing Ethyl 3-bromo-1-(3-chloropyridin-2-yl)-1H-pyrazole-5-carboxylate.

### CROSS-REFERENCE TO RELATED APPLICATION

This application claims the benefit of U.S. Provisional Application No. 62/933,553 filed Nov. 11, 2019.

### FIELD OF INVENTION

This disclosure is directed to novel methods of synthesizing Ethyl 3-bromo-1-(3-chloropyridin-2-yl)-1H-pyrazole-5-carboxylate. Compounds prepared by the methods disclosed herein are useful for preparation of certain anthranilamide compounds that are of interest as insecticides, such as, for example, the insecticides chlorantraniliprole and cyantraniliprole.

### BACKGROUND

Ethyl 3-bromo-1-(3-chloropyridin-2-yl)-1H-pyrazole-5-carboxylate is an intermediate in the production of 3-bromo-N-[4-chloro-2-methyl-6-[(methylamino)carbonyl]phenyl]-1-(3-chloro-2-pyridinyl)-1H-pyrazole-5-carboxamide and 3-Bromo-1-(3-chloropyridin-2-yl)-N-[4-cyano-2-methyl-6-[—N-methyarbaoyl]phenyl]-1H-pyrazole-5-carboxamide.

As disclosed in the patent WO03016283A1, 3-bromo-1-(3-chloropyridin-2-yl)-1H-pyrazole-5-carboxylate is produced from ethyl 3-bromo-1-(3-chloropyridin-2-yl)-4,5-dihydro-1H-pyrazole-5-carboxylate through an oxidation reaction in acetonitrile system in the presence of potassium persulfate using sulfuric acid as catalyst. The reported yield is about 75-80%.

The present disclosure provides novel methods useful for preparing Ethyl 3-bromo-1-(3-chloropyridin-2-yl)-1H-pyrazole-5-carboxylate and derivatives thereof. The benefits of the methods of the present disclosure compared to previous methods are numerous and include improved overall yield, reduced cost, and reduced process hazards.

The disclosed methods provide an overall yield of about 88%.

### BRIEF DESCRIPTION

In one aspect, provided herein is a method of preparing a compound of Formula II, wherein

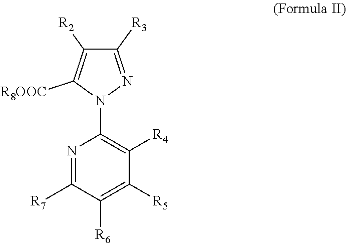

R₈ is selected from hydrogen and C₁-C₄ alkyl; and
each of R₂-R₇ is independently selected from hydrogen, halogen, C₁-C₄ alkyl, and halogenated C₁-C₄ alkyl, the method comprising
I) forming a mixture comprising
   A) a first amount of a compound of Formula I, wherein

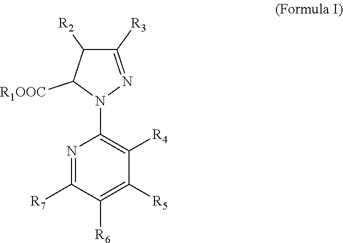

R₁ is selected from hydrogen and C₁-C₄ alkyl; and
each of R₂-R₇ is independently selected from hydrogen, halogen, C₁-C₄ alkyl, and halogenated C₁-C₄ alkyl;
B) an oxidizing agent; and
C) an organic solvent;
II) heating the mixture;
III) adding an acid to the mixture;
IV) adding a second amount of the compound of Formula I to the mixture; and
V) completing the reaction of the mixture.

### DETAILED DESCRIPTION OF THE DISCLOSURE

As used herein, the terms “comprises,” “comprising,” “includes,” “including,” “has,” “having,” “contains”, “containing,” “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Where an invention or a portion thereof is defined with an open-ended term such as “comprising,” it should be readily understood that (unless otherwise stated) the description should be interpreted to also describe such an invention using the terms “consisting essentially of” or “consisting of.”

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As used herein, the term “about” means plus or minus 10% of the value.

The term “eq” refers to the amount of a substance that reacts with (or is equivalent to) an arbitrary amount of another substance in a given chemical reaction.

The term “% assay” refers to the content of the desired compound divided by the total weight of the sample.

The term “halogen”, either alone or in compound words or phrases such as “haloalkyl” or “halogenated alkyl”, includes fluorine, chlorine, bromine or iodine. Further, when used in compound words or phrases such as “haloalkyl” or “halogenated alkyl”, said alkyl may be partially or fully substituted with halogen atoms which may be the same or different.

When a group contains a substituent which can be hydrogen, for example R⁴, then, when this substituent is taken as hydrogen, it is recognized that this is equivalent to said group being unsubstituted.

Certain compounds of this invention can exist as one or more stereoisomers. The various stereoisomers include enantiomers, diastereomers, atropisomers and geometric isomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers.

The embodiments of this disclosure include:

Embodiment 1. A method of preparing a compound of Formula II, wherein

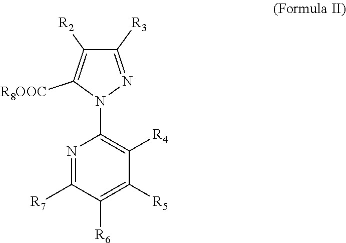

R₈ is selected from hydrogen and C₁-C₄ alkyl; and
each of R₂-R₇ is independently selected from hydrogen, halogen, C₁-C₄ alkyl, and halogenated C₁-C₄ alkyl, the method comprising
I) forming a mixture comprising
   A) a first amount of a compound of Formula I, wherein

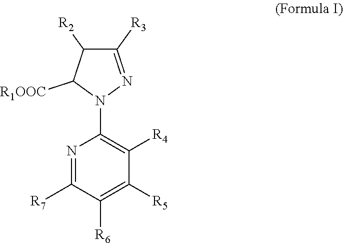

R₁ is selected from hydrogen and C₁-C₄ alkyl; and
each of R₂-R₇ is independently selected from hydrogen, halogen, C₁-C₄ alkyl, and halogenated C₁-C₄ alkyl;
B) an oxidizing agent; and
C) an organic solvent;
II) heating the mixture;
III) adding an acid to the mixture;
IV) adding a second amount of the compound of Formula I to the mixture; and
V) completing the reaction of the mixture.

Embodiment 2. The method of embodiment 1, wherein the oxidizing agent is selected from hydrogen peroxide, organic peroxides, potassium persulfate, sodium persulfate, ammonium persulfate, potassium monopersulfate, sodium monopersulfate, potassium permanganate, and combinations thereof.

Embodiment 3. The method of embodiment 1, wherein the oxidizing agent is potassium persulfate.

Embodiment 4. The method of embodiment 1, wherein the oxidizing agent is present in the mixture in an amount in the range of about 1.0 eq to about 2.0 eq.

Embodiment 5. The method of embodiment 1, wherein the oxidizing agent is present in the mixture in an amount in the range of about 1.3 eq to about 1.7 eq.

Embodiment 6. The method of embodiment 1, wherein the oxidizing agent has a D50 particle size in the range of about 10 μm to about 200 μm.

Embodiment 7. The method of embodiment 1, wherein the oxidizing agent has a D50 particle size in the range of about 20 μm to about 100 μm.

Embodiment 8. The method of embodiment 1, wherein the oxidizing agent has a D50 particle size in the range of about 30 μm to about 80 μm.

Embodiment 9. The method of embodiment 1, wherein the oxidizing agent has a D50 particle size in the range of about 40 μm to about 60 μm.

Embodiment 10. The method of embodiment 1, wherein the solvent is selected from ethers, esters, aprotic organic solvents, and combinations thereof.

Embodiment 11. The method of embodiment 10, wherein the ether is selected from tetrahydrofuran, 2-methyltetrahydrofuran, dioxane, and combinations thereof.

Embodiment 12. The method of embodiment 10, wherein the ester is selected from ethyl acetate, isopropyl acetate, dimethyl carbonate, butyl acetate, and combinations thereof.

Embodiment 13. The method of embodiment 10, wherein the aprotic organic solvent is selected from N,N-dimethylformamide, dimethylacetamide, dimethyl sulfoxide, 1,3-dimethyl-2-imidazolidinone, acetonitrile, and combinations thereof.

Embodiment 14. The method of embodiment 10, wherein the aprotic organic solvent is acetonitrile.

Embodiment 15. The method of embodiment 1, wherein the first amount of the compound of Formula I is about 5 wt % to about 40 wt % of the total amount of the compound of Formula I.

Embodiment 16. The method of embodiment 1, wherein the first amount of the compound of Formula I is present in the mixture in a concentration in the range of about 10 wt % to about 30 wt % of the total amount of the compound of Formula I.

Embodiment 17. The method of embodiment 1, wherein the acid is selected from inorganic acids, organic acids, and combinations thereof.

Embodiment 18. The method of embodiment 17, wherein the organic acid is selected from acetic acid, propanoic acid, p-toluenesulfonic acid, benzoic acid, and combinations thereof.

Embodiment 19. The method of embodiment 17, wherein the inorganic acid is selected from sulfuric acid, phosphoric acid, oleum, hydrobromic acid, hydrochloric acid, and combinations thereof.

Embodiment 20. The method of embodiment 17, wherein the inorganic acid is sulfuric acid.

Embodiment 21. The method of embodiment 1, wherein the acid is present in the mixture in an amount in the range of about 0.05 eq to about 1.5 eq.

Embodiment 22. The method of embodiment 1, wherein the acid is present in the mixture in an amount less than about 0.2 eq.

Embodiment 23. The method of embodiment 1, wherein the method step of forming the mixture occurs at a temperature in the range of about 0° C. to about 60° C.

Embodiment 24. The method of embodiment 1, wherein the method step of forming the mixture occurs at a temperature in the range of about 15° C. to about 35° C.

Embodiment 25. The method of embodiment 1, wherein the method step of forming the mixture occurs at room temperature.

Embodiment 26. The method of embodiment 1, wherein the method step of heating the mixture increases the temperature of the mixture to a temperature in the range of about 50° C. to about 82° C.

Embodiment 27. The method of embodiment 1, wherein the method step of heating the mixture increases the temperature of the mixture to a temperature in the range of about 55° C. to about 65° C.

Embodiment 28. The method of embodiment 1, wherein the method step of adding a second amount of the compound of Formula I to the mixture occurs at a temperature in the range of about 50° C. to about 82° C.

Embodiment 29. The method of embodiment 1, wherein the method step of adding a second amount of the compound of Formula I to the mixture occurs at a temperature in the range of about 65° C. to about 82° C.

Embodiment 30. The method of embodiment 1, wherein the method step of adding a second amount of the compound of Formula I comprises discretely adding the second amount of the compound of Formula I.

Embodiment 31. The method of embodiment 1, wherein the method step of adding a second amount of the compound of Formula I comprises continuously adding the second amount of the compound of Formula I.

Embodiment 32. The method of embodiment 1, wherein the method step of adding a second amount of the compound of Formula I comprises dropwise addition of the second amount of the compound of Formula I.

Embodiment 33. The method of embodiment 1, wherein the method step of adding a second amount of the compound of Formula I occurs over a period of time in the range of about 3 hours to about 7 hours.

Embodiment 34. The method of embodiment 1, wherein the method step of adding a second amount of the compound of Formula I occurs over a period of time in the range of about 3.5 hours to about 4.5 hours.

Embodiment 35. The method of embodiment 1, wherein the second amount of the compound of Formula I is greater than the first amount of the compound of Formula I.

Embodiment 36. The method of embodiment 1, wherein the second amount of the compound of Formula I is at least twice the first amount of the compound of Formula I.

Embodiment 37. The method of embodiment 1, wherein the second amount of the compound of Formula I is about 70 wt % of the total amount of the compound of Formula I.

Embodiment 38. The method of embodiment 1, wherein the first amount of the compound of Formula I is about 30 wt % of the total amount of the compound of Formula I.

Embodiment 39. The method of embodiment 1, wherein at least one method step further comprises detecting the O₂ content of the mixture with an oxygen sensor.

Embodiment 40. The method of embodiment 1, wherein the method step of heating the mixture occurs in the absence of O₂.

Embodiment 41. The method of embodiment 1, wherein the method produces less than about 0.5 wt % 02.

Embodiment 42. The method of embodiment 1, wherein the method does not produce O₂.

Embodiment 43. The method of embodiment 1, wherein at least one method step further comprises stirring the mixture.

In one aspect, Ethyl 3-bromo-1-(3-chloropyridin-2-yl)-1H-pyrazole-5-carboxylate is prepared according to a method represented by Scheme 1.

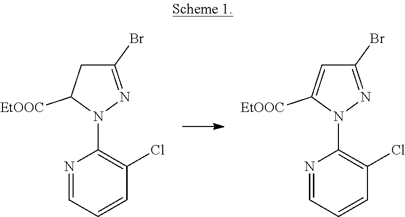 [SMILES: CCOC(=O)C1CC(Br)=NN1c1ncccc1Cl ; CCOC(=O)c1cc(Br)nn1-c1ncccc1Cl]

In one aspect, a compound of Formula II is prepared according to a method represented by Scheme 2. The R groups are as defined anywhere in this disclosure.

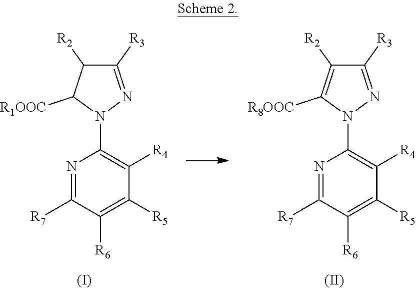

This aspect includes forming a mixture comprising a first amount of a compound of Formula I, an oxidizing agent, and an organic solvent, heating the mixture, adding an acid to the mixture, adding a second amount of the compound of Formula I to the mixture, and completing the reaction of the mixture.

In one embodiment, the oxidizing agent is selected from hydrogen peroxide, organic peroxides, potassium persulfate, sodium persulfate, ammonium persulfate, potassium monopersulfate, sodium monopersulfate, potassium permanganate, and combinations thereof. In another embodiment, the oxidizing agent is potassium persulfate. In one embodiment, the oxidizing agent is present in the mixture in an amount in the range of about 1.0 eq to about 2.0 eq. In another embodiment, the oxidizing agent is present in the mixture in an amount in the range of about 1.3 eq to about 1.7 eq. In one embodiment, the oxidizing agent has a D50 particle size in the range of about 10 μm to about 200 μm. In another embodiment, the oxidizing agent has a D50 particle size in the range of about 20 μm to about 100 μm. In another embodiment, the oxidizing agent has a D50 particle size in the range of about 30 μm to about 80 μm. In another embodiment, the oxidizing agent has a D50 particle size in the range of about 40 μm to about 60 μm.

In one embodiment, the solvent is selected from ethers, esters, aprotic organic solvents, and combinations thereof. In another embodiment, the solvent is an ether selected from tetrahydrofuran, 2-methyltetrahydrofuran, dioxane, and combinations thereof. In another embodiment, the solvent is an ester selected from ethyl acetate, isopropyl acetate, dimethyl carbonate, butyl acetate, and combinations thereof. In one embodiment, the solvent is an aprotic organic solvent selected from N,N-dimethylformamide, dimethylacetamide, dimethyl sulfoxide, 1,3-dimethyl-2-imidazolidinone, acetonitrile, and combinations thereof. In another embodiment, the solvent is acetonitrile.

In one embodiment, the first amount of the compound of Formula I is about 5 wt % to about 40 wt % of the total amount of the compound of Formula I. In another embodiment, the first amount of the compound of Formula I is about 10 wt % to about 30 wt % of the total amount of the compound of Formula I.

In one embodiment, the acid is selected from inorganic acids, organic acids, and combinations thereof. In another embodiment, the acid is an organic acid selected from acetic acid, propanoic acid, p-toluenesulfonic acid, benzoic acid, and combinations thereof. In another embodiment, the acid is an inorganic acid selected from sulfuric acid, phosphoric acid, oleum, hydrobromic acid, hydrochloric acid, and combinations thereof. In another embodiment, the acid is sulfuric acid. In one embodiment, the acid is present in the mixture in an amount in the range of about 0.05 eq to about 1.5 eq. In another embodiment, the acid is present in the mixture in an amount less than about 0.2 eq.

In one embodiment, the method step of forming the mixture occurs at a temperature in the range of about 0° C. to about 60° C. In another embodiment, the method step of forming the mixture occurs at a temperature in the range of about 15° C. to about 35° C. In another embodiment, the method step of forming the mixture occurs at room temperature. In one embodiment, the method step of forming the mixture comprises stirring the mixture

In one embodiment, the method step of heating the mixture increases the temperature of the mixture to a temperature in the range of about 50° C. to about 82° C. In another embodiment, the method step of heating the mixture increases the temperature of the mixture to a temperature in the range of about 55° C. to about 65° C. In one embodiment, the method step of heating the mixture comprises stirring the mixture

In one embodiment, the method step of adding a second amount of the compound of Formula I to the mixture occurs at a temperature in the range of about 50° C. to about 82° C. In another embodiment, the method step of adding a second amount of the compound of Formula I to the mixture occurs at a temperature in the range of about 50° C. to about 82° C. In one embodiment, the method step of adding a second amount of the compound of Formula I comprises discretely adding the second amount of the compound of Formula I. In one embodiment, the method step of adding a second amount of the compound of Formula I comprises continuously adding the second amount of the compound of Formula I. In another embodiment, the method step of adding a second amount of the compound of Formula I comprises dropwise addition of the second amount of the compound of Formula I. In one embodiment, the method step of adding a second amount of the compound of Formula I occurs over a period of time in the range of about 3 hours to about 7 hours. In another embodiment, the method step of adding a second amount of the compound of Formula I occurs over a period of time in the range of about 3.5 hours to about 4.5 hours. In one embodiment, the method step of adding a second amount of the compound of Formula I comprises stirring the mixture

In one embodiment, the second amount of the compound of Formula I is greater than the first amount of the compound of Formula I. In another embodiment, the second amount of the compound of Formula I is at least twice the first amount of the compound of Formula I. In one embodiment, the second amount of the compound of Formula I is about 70 wt % of the total amount of the compound of Formula I. In another embodiment, the first amount of the compound of Formula I is about 30 wt % of the total amount of the compound of Formula I. In one embodiment, the second amount of the compound of Formula I is about 80 wt % of the total amount of the compound of Formula I. In another embodiment, the first amount of the compound of Formula I is about 20 wt % of the total amount of the compound of Formula I. In one embodiment, the second amount of the compound of Formula I is about 90 wt % of the total amount of the compound of Formula I. In another embodiment, the first amount of the compound of Formula I is about 10 wt % of the total amount of the compound of Formula I. In one embodiment, the second amount of the compound of Formula I is about 95 wt % of the total amount of the compound of Formula I. In another embodiment, the first amount of the compound of Formula I is about 5 wt % of the total amount of the compound of Formula I.

In one embodiment, at least one method step further comprises detecting the O₂ content of the mixture with an oxygen sensor. In one embodiment, the method step of heating the mixture occurs in the absence of O₂. In one embodiment, the method produces less than about 0.5 wt % 02. In another embodiment, the method does not produce O₂. Low and/or nondetectable O₂ production increases process safety.

### Examples

Without further elaboration, it is believed that one skilled in the art using the preceding description can utilize the present invention to its fullest extent. The following Examples are, therefore, to be construed as merely illustrative, and not limiting of the disclosure in any way whatsoever. The starting material for the following Examples may not have necessarily been prepared by a particular preparative run whose procedure is described in other Examples. It also is understood that any numerical range recited herein includes all values from the lower value to the upper value. For example, if a range is stated as 10-50, it is intended that values such as 12-30, 20-40, or 30-50, etc., are expressly enumerated in this specification. These are only examples of what is specifically intended, and all possible combinations of numerical values between and including the lowest value and the highest value enumerated are to be considered to be expressly stated in this application.

### Comparative Example 1. Method of Monitoring Oxygen Generation

153 g potassium persulfate, 450 g acetonitrile, and other starting materials are added to a 2 L flask equipped with a condenser, thermometer, dropping funnel, and nitrogen input pipe. An oxygen sensor is set up over the condenser. The air in the reactor is replaced with nitrogen flowing at a rate of 0.55 L/min. When the oxygen sensor shows 0.0% oxygen level, the mixture is heated to 60° C. Then 4.0 g sulfuric acid is added to the mixture and the mixture is kept heating to reflux. During the whole process, the oxygen level is monitored and recorded.

### Comparative Example 2. Single Addition of Ethyl 3-Bromo-1-(3-Chloropyridin-2-Yl)-4,5-Dihydro-1H-Pyrazole-5-Carboxylate at the Start of the Reaction

174 g potassium persulfate (1.6 eq), 780 g 17% ethyl 3-bromo-1-(3-chloropyridin-2-yl)-4,5-dihydro-1H-pyrazole-5-carboxylate in acetonitrile, and 40 g of concentrated sulfuric acid (1.0 eq) are added to a reactor. The reaction mixture is stirred and heated to 65° C. The reaction shows obvious exothermic release and thus self-heats to reflux. The reaction mixture is kept at reflux for another 3-4 hours to complete the reaction. No oxygen is detected throughout the process. 350 g water is added to the mixture at 70° C. and the phase is split with potassium bisulfate to remove waste water. After stripping solvent and subsequent crystallization, about 107 g ethyl 3-bromo-1-(3-chloropyridin-2-yl)-1H-pyrazole-5-carboxylate dry product is obtained with about 96% assay and about 78% yield.

### Example 1. Addition of Ethyl 3-Bromo-1-(3-Chloropyridin-2-Yl)-4,5-Dihydro-1H-Pyrazole-5-Carboxylate at the Start of the Reaction and after the Start of the Reaction

100 g 40% ethyl 3-bromo-1-(3-chloropyridin-2-yl)-4,5-dihydro-1H-pyrazole-5-carboxylate in acetonitrile, 153 g potassium persulfate (1.4 eq), and 450 g acetonitrile are added to a reactor. The reaction mixture is stirred and heated to 60° C. and then 4.0 g concentrated sulfuric acid (0.1 eq) is added to the mixture. The reaction mixture is kept at reflux for 1 hour and then 230 g 40% ethyl 3-bromo-1-(3-chloropyridin-2-yl)-4,5-dihydro-1H-pyrazole-5-carboxylate in acetonitrile is added dropwise during a period of 3-5 hours. After all materials are added, the reaction mixture is kept at reflux for another 1-2 hours to complete the reaction. No oxygen is detected throughout the process. 350 g water is added to the mixture at 70° C. and the phase is split with potassium bisulfate to remove waste water. After stripping solvent and subsequent crystallization, about 120 g ethyl 3-bromo-1-(3-chloropyridin-2-yl)-1H-pyrazole-5-carboxylate dry product is obtained with about 97% assay and about 88% yield.

This written description uses examples to illustrate the present disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A method of preparing a compound of Formula II, wherein 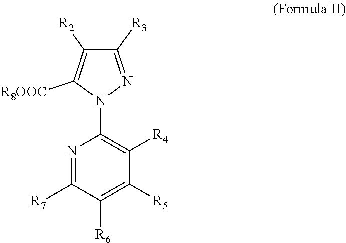
R₈ is selected from hydrogen and C₁-C₄ alkyl; and
each of R₂-R₇ is independently selected from hydrogen, halogen, C₁-C₄ alkyl, and halogenated C₁-C₄ alkyl, the method comprising
I) forming a mixture comprising
A) a first amount of a compound of Formula I, wherein 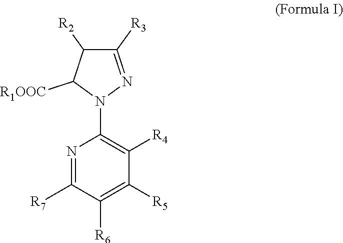
R₁ is selected from hydrogen and C₁-C₄ alkyl; and
each of R₂-R₇ is independently selected from hydrogen, halogen, C₁-C₄ alkyl, and halogenated C₁-C₄ alkyl;
B) an oxidizing agent; and
C) an organic solvent;
II) heating the mixture;
III) adding an acid to the mixture;
IV) adding a second amount of the compound of Formula I to the mixture; and
V) completing the reaction of the mixture.

2. The method of claim 1, wherein the oxidizing agent is selected from hydrogen peroxide, organic peroxides, potassium persulfate, sodium persulfate, ammonium persulfate, potassium monopersulfate, sodium monopersulfate, potassium permanganate, and combinations thereof.

3. The method of claim 1, wherein the oxidizing agent is present in the mixture in an amount in the range of about 1.0 eq to about 2.0 eq.

4. The method of claim 1, wherein the oxidizing agent has a D50 particle size in the range of about 10 μm to about 200 μm.

5. The method of claim 1, wherein the solvent is selected from ethers, esters, aprotic organic solvents, and combinations thereof.

6. The method of claim 1, wherein the first amount of the compound of Formula I about 5 wt % to about 40 wt % of the total amount of the compound of Formula I.

7. The method of claim 1, wherein the acid is selected from inorganic acids, organic acids, and combinations thereof.

8. The method of claim 1, wherein the acid is present in the mixture in an amount in the range of about 0.05 eq to about 1.5 eq.

9. The method of claim 1, wherein the acid is present in the mixture in an amount less than about 0.2 eq.

10. The method of claim 1, wherein the method step of forming the mixture occurs at a temperature in the range of about 0° C. to about 60° C.

11. The method of claim 1, wherein the method step of heating the mixture increases the temperature of the mixture to a temperature in the range of about 50° C. to about 82° C.

12. The method of claim 1, wherein the method step of adding a second amount of the compound of Formula I comprises discretely adding the second amount of the compound of Formula I.

13. The method of claim 1, wherein the method step of adding a second amount of the compound of Formula I comprises continuously adding the second amount of the compound of Formula I.

14. The method of claim 1, wherein the method step of adding a second amount of the compound of Formula I comprises dropwise addition of the second amount of the compound of Formula I.

15. The method of claim 1, wherein the method step of adding a second amount of the compound of Formula I occurs over a period of time in the range of about 3 hours to about 7 hours.

16. The method of claim 1, wherein the second amount of the compound of Formula I is greater than the first amount of the compound of Formula I.

17. The method of claim 1, wherein the second amount of the compound of Formula I is at least twice the first amount of the compound of Formula I.

18. The method of claim 1, wherein the second amount of the compound of Formula I is at least 70 wt % of the total amount of the compound of Formula I.

19. The method of claim 1, wherein the first amount of the compound of Formula I is less than 30 wt % of the total amount of the compound of Formula I.

20. The method of claim 1, wherein at least one method step further comprises stirring the mixture.
